(19) United States
(12) Patent Application Publication
(10) Pub. No.: US 2011/0160792 A1
(43) Pub. Date: Jun. 30, 2011

(54) Method And Device For Determination Of Efficacy Of Cardiac Resynchronization Pacing Utilizing Simultaneous RV And LV Electroanotomic Cardiac Phase Motion Mapping

(76) Inventor: Robert S. Fishel, Delray Beach, FL (US)

(21) Appl. No.: 12/974,831

(22) Filed: Dec. 21, 2010

Related U.S. Application Data
(60) Provisional application No. 61/289,918, filed on Dec. 23, 2009.

(51) Int. Cl. A61N 1/37

(52) U.S. Cl. 607/27

(57) ABSTRACT

The current invention describes a method and system to measure and validate the efficacy of cardiac resynchronization therapy pacing using electroanatomical position and motion sensing during the various phases of the cardiac cycle. In this method, electroanatomical position and motion sensors are utilized with sensing from the tip of both right ventricular pacing lead and left ventricular pacing lead. An operator can therefore obtain data not currently available to an implanter with current technology. This data includes the physical distance between both leads and the relative motion of both leads during cardiac resynchronization therapy biventricular pacing. If good lead positioning for both the right ventricular lead and left ventricular lead has been obtained, then the operator will be able to demonstrate good synchronization of the cardiac cycle.

### REFERENCE TO RELATED APPLICATIONS

This Application claims priority under 35 U.S.C. §119(e) to U.S. Provisional Application 61/289,918, filed on Dec. 23, 2009, the contents of which are herein incorporated by reference.

### FIELD OF THE INVENTION

The instant invention relates to cardiac pacing or stimulation systems, and more particularly to cardiac resynchronization therapy systems and methods for determining the optimal placement of cardiac resynchronization therapy leads within the right ventricular and left ventricular areas of the heart to ensure synchronization of each of the heart chambers after pacing events.

### BACKGROUND OF THE INVENTION

The heart is a vital aspect of human physiology responsible for transporting and exchanging substances between the environment and the cells that make up the various tissues and organs. The main function of the heart is to pump blood through the circulatory system using coordinated, rhythmic contractions. Such contractions are based on a complex series of electrocardio and mechanocardio events. Briefly, during periods of contractions, the heart muscles pump blood out through the arteries. During periods of relaxation, the heart fills with deoxygenated blood in the right ventricles, and oxygenated blood in the left ventricles. The main driving force for contraction and relaxation rhythms are the cardioelectrical events. The sinoatrial node (SA node), often called the natural pacemaker, is part of the heart's natural conduction system and is made of specialized cells that are capable of generating electrical signals. Electrical events are generated in the SA node, eventually spreading to the rest of the heart via nodal tissue pathways which coordinate the events of the cardiac cycle. The electrical signals generated through the pathway cause the heart muscles to squeeze and release in a coordinated, rhythmic sequence that result in drawing blood into the heart chambers and forcing blood out of the chambers.

Despite advances in the understanding and treatment of cardiac diseases, heart failure remains a major issue and cause of concern for physicians. It is estimated that heart failure affects millions of individuals in the United States, and tens of millions of people worldwide. Those suffering from congestive heart failure (CHF) often have a poor quality of life and have high mortality rates. One promising method which has gained a lot of attention for treating CHF over the last ten years is cardiac resynchronization therapy. Cardiac resynchronization therapy (CRT) pacing is a technique allowing for pacing of both the right ventricle (RV) and the left ventricle (LV) simultaneously to allow for synchronous contraction of both ventricles during a cardiac cycle. In patients suffering from dysynchronous heart functions, such as those who acquire a left bundle branch block, the native conduction system of the left ventricle no longer conducts heart beats delivered to the AV node from the atria. In this situation, the left ventricle follows the right ventricle in its contraction, with the left ventricle contraction occurring via slow cell to cell conduction as opposed to the much faster conduction present in the setting of an intact left bundle branch. This situation causes cardiac dysynchrony and there is no longer a coordinated contraction between the right ventricle and left ventricle during any given heart beat. In cardiac resynchronization therapy, an additional left ventricle pacing lead is delivered to a region of the left ventricle either via an epicardial or endocardial approach and left ventricle pacing is provided at the same time as right ventricle pacing, allowing for synchronous contraction of both ventricles. Cardiac resynchronization therapy pacing will typically improve a patients' ejection fraction and decrease his symptoms of congestive heart failure if cardiac resynchronization therapy is performed correctly.

In order for cardiac resynchronization therapy to be performed correctly, both the right ventricle pacing lead and the left ventricle pacing lead need to be physically separated in three-dimensional space. If these electrodes are placed proximate to each other, then resynchronization will not occur. Clinical data suggests that the further apart these electrodes are physically, the better the resynchronization will be. In a dysynchronous ventricular contraction, the left ventricle free wall tends to move away from the right ventricle free wall when the right ventricle free wall is contracting towards the interventricular septum. In a synchronous contraction, both the left ventricle free wall and the right ventricle free wall will move towards each other and towards the interventricular septum. In addition to the physical separation of both leads needed for ventricular resynchronization, good ventricular resynchronization requires that during pacing of both leads, both free walls move toward each other and the interventricular septum.

Cardiac electroanatomical mapping utilizes a variety of techniques which allow for three-dimensional tracking of electrode positions inside the heart. In addition to positioning, electroanatomical mapping allows for measuring an objects pitch, yaw, roll and direction of motion inside the heart or other body structure. Two current techniques to allow for this type of motion/location tracking are available: one utilizes external magnetic fields as positional references, the other uses an internal or body surface reference and changes in either lead impedance or current to track position and motion. Both techniques allow for precise tracking of a pacing leads position and its motion.

### DESCRIPTION OF THE PRIOR ART

Several methods for determining lead position in cardiac resynchronization therapy have been described in the prior art. For example, U.S. Pat. No. 7,751,882 discloses methods and systems for determining a location for delivering cardiac pacing pulses to the myocardium of a heart chamber. Temporal information pertaining to both myocardial electrical activity and myocardial mechanical activity at a plurality of locations relative to the myocardium of the heart chamber is obtained. For each location, the temporal difference between a feature of the electrical activity and a feature of the mechanical activity is compared. A stimulation electrode is then positioned at one of the locations based on the comparison.

U.S. Pat. No. 7,343,196 discloses a method which includes obtaining non-invasive acquisition data from a medical imaging system and generating a three-dimensional model of the left ventricle and thoracic wall of the patient from these data. One or more left ventricle anatomical landmarks are identified on the three-dimensional model, and saved views of the three-dimensional model are registered on an interventional system. One or more of the registered saved views are visualized with the interventional system, and at least one suitable region on the left ventricle wall is identified for epicardial lead placement.

U.S. Pat. No. 6,978,184 describes an optimization method for cardiac resynchronization therapy. The method includes selecting a location to place the leads of a cardiac pacing device, collecting seismocardiographic (SCG) data corresponding to heart motion during paced beats of a patient's heart, determining hemodynamic and electrophysiological parameters based on the SCG data, repeating the preceding steps for another lead placement location, and selecting a lead placement location that provides the best cardiac performance by comparing the calculated hemodynamic and electrophysiological parameters for each different lead placement location.

United States Patent Application 2004/0181260 describes a method for optimizing patient outcome from cardiac resynchronization therapy. The method includes a process by which sets of dynamic cardiopulmonary dependent variables are measured during steady-state conditions, displayed, and translated into quantitative and qualitative measurements while the independent variables of CRT, device lead placement and atrial-ventricular and interventricular delay settings of biventricular pacemaker systems, are altered by a physician. In combination with visual observation and computer-assisted ranking of the dependent variables, a physician can utilize the resulting information to render decisions on the optimal choice of the independent variables.

United States Patent Application 2009/0306732 describes a method which includes providing a mechanical activation time for a myocardial location, the location defined at least in part by an electrode, and the mechanical activation time determined at least in part by movement of the electrode. The method further includes providing an electrical activation time for the myocardial location and determining an electromechanical delay for the myocardial location based on the difference between the mechanical activation time and the electrical activation time.

### SUMMARY OF THE INVENTION

The current invention embodies a method and system to measure and validate the efficacy of cardiac resynchronization therapy pacing using electroanatomical position and motion sensing during the various phases of the cardiac cycle. In this method, electroanatomical position and motion sensors are utilized with sensing from the tip of both the right ventricular pacing lead and the left ventricular pacing lead. Utilizing this technique, a surgeon can obtain data not currently available to an implanter with current technology. This data includes the physical distance between both leads and the relative motion of both leads for cardiac resynchronization therapy biventricular pacing. If good lead positioning for both the right ventricular lead and left ventricular lead has been obtained, then the surgeon will be able to demonstrate that there is greater than a pre-defined minimal physical distance between the leads after implantation. In addition, good resynchronization will be associated with a change in the relative dynamic movement of the left ventricle free wall versus the right ventricular pacing lead and good resynchronization should be associated with movement of both electrodes towards each other, versus movement of the right ventricular and left ventricular lead away from each other should poor lead positioning have been obtained. This method will therefore allow the operator to demonstrate in real time both that adequate lead separation has objectively and quantitatively been achieved and that resynchronization is occurring with these respective lead positions.

One illustrative embodiment of the method for determining the efficacy of cardiac resynchronization pacing utilizing simultaneous right ventricle and left ventricle electroanatomic cardiac phase motion mapping includes the steps of:

Step 1: Using at least 2 cardiac resynchronization therapy pacing leads each having one or more sensors for obtaining data related to lead placement efficiency determining characteristics into a patient's heart; the leads being electrically coupled to a cardiac resynchronization therapy pacing device for delivery of a cardiac resynchronization therapy event.

Step 2: Observing one or more efficiency determining characteristics for each of the leads for at least once cycle of said patient's cardiac rhythm.

Step 3: Providing a pacing event to the heart from the cardiac resynchronization therapy pacing device.

Step 4: Observing one or more efficiency determining characteristics for each of the leads for at least one cardiac resynchronization therapy cycle of the cardiac rhythm.

Step 5: Determining if the position of each of the leads relative to each other provides efficient cardiac resynchronization therapy to achieve synchronized cardiac rhythms.

Step 6: Moving at least one of the leads to a second position in the heart if the cardiac resynchronization therapy does not result in synchronized cardiac rhythms.

Step 7: Providing a second pacing event to the at least one repositioned leads.

Step 8: Observing one or more efficiency determining characteristics for the repositioned leads for at least one cycle of the cardiac rhythm.

Step 9: Determining if the repositioned leads provide efficient cardiac resynchronization therapy to achieve synchronized cardiac rhythms.

Step 10: Repeating steps 6-9 until synchronized cardiac rhythms are achieve.

An alternative illustrative method according to the instant invention includes the steps of: introducing a first cardiac resynchronization therapy lead having one or more sensors for obtaining cardiac resynchronization therapy efficiency characteristics, the first lead being electrically coupled to the right ventricle of a patient's heart; introducing a second cardiac resynchronization therapy lead having one or more sensors for obtaining cardiac resynchronization therapy efficiency characteristics, the second lead being electrically coupled to the left ventricle of said patient's heart; obtaining efficiency characteristics of the first lead and the second lead prior to providing a cardiac resynchronization therapy pacing event; providing a cardiac resynchronization therapy pacing event; obtaining a second set of efficiency determining characteristics of the first and second leads after the pacing event; and determining if the cardiac resynchronization therapy pacing event correctly resynchronized the patient's heart.

If the cardiac resynchronization therapy pacing event does not result in synchronous cardiac rhythms, the method in accordance with the instant invention further includes the steps of: moving at least one lead to another position in the patient's heart if the cardiac resynchronization therapy pacing event did not result in cardiac resynchronization; obtaining additional cardiac resynchronization therapy pacing efficiency characteristics for repositioning said leads; delivering additional cardiac resynchronization therapy pacing event; determining if repositioning of the leads results in resynchronization of the patient's heart; repeating steps until each of the leads are placed in a position which results in cardiac resynchronization.

Accordingly, it is an objective of the instant invention to provide a method for determining the efficacy of cardiac resynchronization pacing utilizing simultaneous right ventricle and left ventricle electroanatomic cardiac phase motion mapping.

It is a further objective of the instant invention to provide a system for determining the efficacy of cardiac resynchronization pacing utilizing simultaneous right ventricle and left ventricle electroanatomic cardiac phase motion mapping.

It is yet another objective of the instant invention to provide a method to validate the efficacy of cardiac resynchronization therapy pacing using electroanatomical position and motion sensing during the various phases of the cardiac cycle.

It is a still further objective of the invention to provide a method to measure the efficacy of cardiac resynchronization therapy pacing using electroanatomical position and motion sensing during the various phases of the cardiac cycle.

It is yet another objective to provide a method to validate and measure the efficacy of cardiac resynchronization therapy pacing using electroanatomical position and motion sensing during the various phases of the cardiac cycle using physical distance between lead placement data.

It is still another objective to provide a method to validate and measure the efficacy of cardiac resynchronization therapy pacing using electroanatomical position and motion sensing during the various phases of the cardiac cycle using data which indicates the relative motion of lead placement.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is an illustrative example of the system for determining the efficacy of cardiac resynchronization pacing utilizing simultaneous right ventricle and left ventricle electroanatomic cardiac phase motion mapping in accordance with the instant invention;

FIG. 2 illustrates an alternative embodiment of a cardiac resynchronization therapy lead in accordance with the instant invention;

FIG. 3 is a schematic illustration of a cardiac resynchronization therapy lead placement in accordance with the instant invention;

FIG. 4 is a flow chart of an illustrative example of a method for determining the efficacy of cardiac resynchronization pacing utilizing simultaneous right ventricle and left ventricle electroanatomic cardiac phase motion mapping in accordance with the instant invention.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

During normal rhythms of the heart beat, the left ventricles (LV) and the right ventricles (RV) contract and relax. Such movements result in the walls of each of the atriums moving inward during each beat. When viewing the contractions and relaxations of the heart under synchronous contractions, this normal physiology results in the normal movement toward the center portion of the heart. For those patients who suffer from cardiac conduction abnormalities, such movement is not viewed. These patients suffer from cardiac dysynchronization. For example, those suffering from left bundle branch block (LBBB); the right side generally functions normally. As each contraction/relaxation occurs, the right ventricle moves toward the center. However, as a result of the blockage in the left vertical, the left ventricle does not contract at the correct time, and moves away from the center and wobbles. Eventually the left ventricle does contract, pushing some of the blood out. Because both sides do not squeeze at the same time, blood in the ventricles typically moves around within the heart with only a portion of the blood being ejected, resulting in cardiac dysynchrony. To correct the improper timing of the LV/RV contractions, cardiac resynchronization therapy pacing (CRT) is utilized to reduce dysynchrony and maximize ejection fraction of the blood. The instant invention utilizes the synchronization of the LV and RV seen in normal heart beating movements, for providing a method which determines the efficacy of the lead placement during CRT.

FIG. 1 illustrates a cardiac resynchronization therapy device 10 in accordance with the method and system for determining the efficacy of cardiac resynchronization pacing utilizing simultaneous right ventricle and left ventricle electroanatomic cardiac phase motion mapping in accordance with the instant invention. The device generally contains a control unit 12 and a plurality of leads 14, 16, and 18 in electrical communication with the control unit 12. Each of the leads 14, 16, and 18 may contain at least one electrode 19. While illustrated herein as a distal tip electrode, electrode 19 may be placed anywhere along the lead body and may also be a ring electrode. The control unit 12 may contain a variety of subunits for optimal use. The control unit must contain at least several subunits which generate electrical, or pacing events, including a battery 20 and a computer processing subunit 22 with memory capabilities to control the timing of the pulse generation and process information from other subunits, thereby coordinating other functions. Other subunits 24, 26 may be included to provide, for example, other types of stimulus, such as defibrillation, left ventricular stimulation, right ventricular stimulation, atrial stimulation, or to detect other types of arrhythmias. In this manner, the cardiac resynchronization therapy device can provide other functionality, such as slowing down abnormally fast heart rhythms, preventing abnormally slow heart rhythms, recording the patient's heart rate and rhythms, in addition to synchronizing the left and right ventricles. Optionally, the system may include a processing device 28, such as a computer with memory, a display device 30 such as a monitor, and data input devices such as a keyboard and mouse. The control unit 12 may be connected via a cable 33 to the processing device 28 for obtaining and processing data and displaying such data on a display device 30. Although only one is illustrated, each of the leads 14, 16, and 18 may be connected to the processing device 28 via cable 34. In this manner, processing device 28 may collect, interpret, and process data from each of the leads.

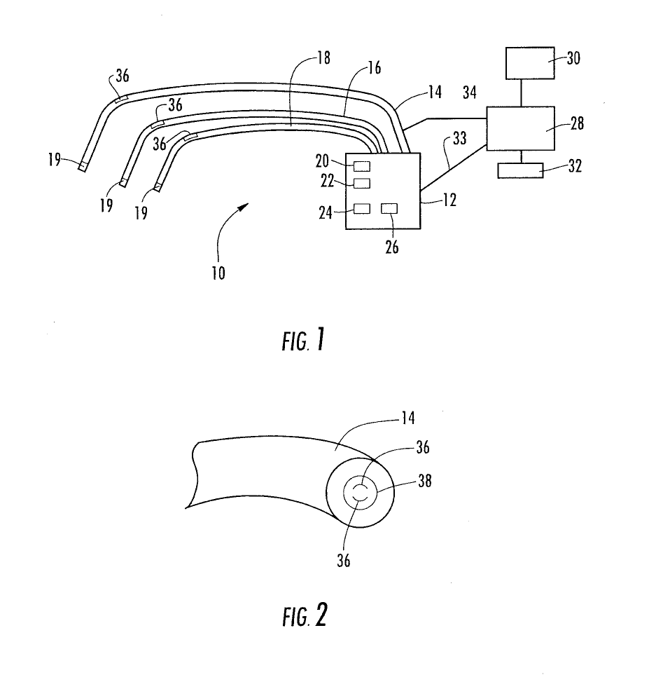

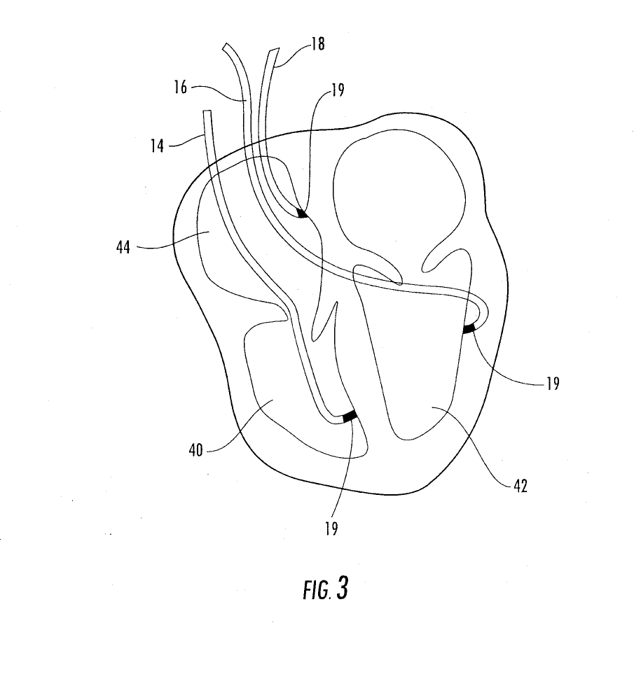

In addition to the electrodes, the leads 14, 16, and may contain one or more sensors 36. The sensors 36 are designed to obtain data regarding one or more cardiac resynchronization therapy efficiency determining characteristics. For example, each of the leads may contain sub-millimeter sized sensors which can obtain data relating to the three-dimensional positioning and orientation of the sensor, calculated in real time and graphically projected on a display image. The sensors preferably provide data which can be used to calculate the distance between each of the leads when each of the leads are electrically coupled to the heart, measure the leads yaw (rotation about a Y-axis), roll (rotation about a X-axis), and pitch (rotation about a Z-axis), calculate placement of the pacing leads within the heart, and also provide information for determining the relative motion of one lead to one of the other leads. The data generated can be projected as two-dimensional or three-dimensional images that may be overlaid or inserted onto other images, such as fluoroscopy, CT scans, ultrasound, or echocardiography images. Alternatively, data relating to the sensors may be viewed singularly on any viewing device well know in the art, such as but not limited to a computer monitor. Sensors, such as those manufactured by Mediguide and used in their intra body navigation devices or their medical positioning systems devices, such as the GMPS™ system which utilizes miniature sensors to track in real time, are known in the art and can be used. Alternatively, the sensors 36 can be inserted within each of the leads 14, 16, or 18 through the use of a stylet, see FIG. 2.

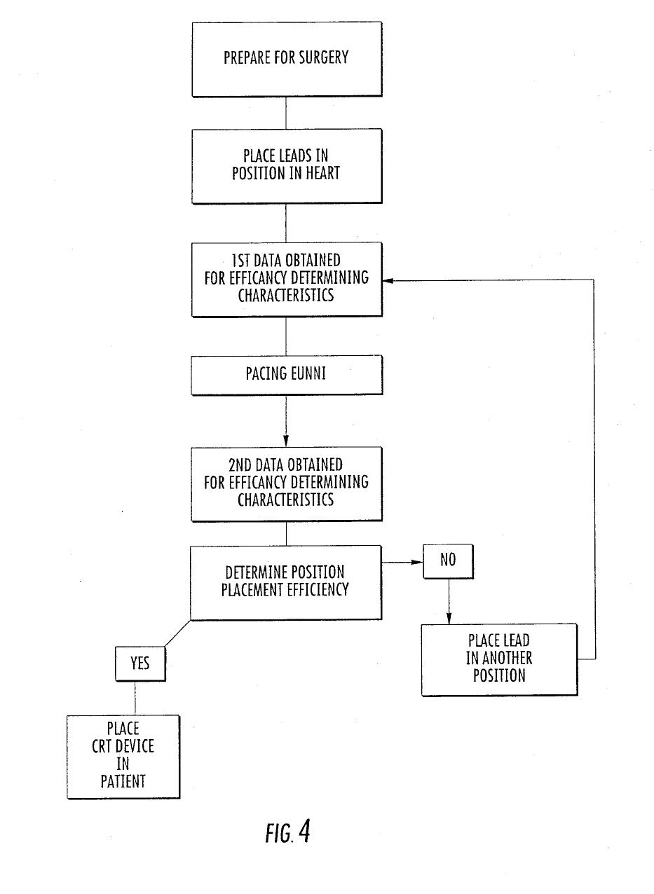

Referring to FIG. 3, leads 14, 16, and 18 are illustrated coupled to the patient's heart in a plurality of positions. For example, lead 14 is placed within the right ventricle 40. The lead 14 therefore provides pacing and sensing of the right ventricle. Lead 16 is illustrated electrically coupled to the left ventricle 42. While the lead 16 may be inserted directly into the left ventricle, it is preferable that left ventricle pacing and sensing can be obtained by coupling the lead 16 to the coronary sinus branch. As used herein, the coronary sinus branch is defined to include the left ventricle vasculature, including, but not limited to, any portion of the coronary sinus, great cardiac vein, left marginal vein, left posterior ventricular vein, middle cardiac vein, and/or the small vein, or any other vein accessible by the coronary sinus. The third lead 18 is electrically coupled to the right atrium, thereby providing pacing and sensing to this region.

Referring to FIG. 3, an overview of the method for determining the efficacy of cardiac resynchronization pacing utilizing simultaneous right ventricular and left ventricular electroanatomic cardiac phase motion mapping is provided. While FIG. 3 describes an illustrative example of the method, steps may be omitted, performed in a different order, or new steps may be added in accordance with the instant invention. A patient is prepared for surgical implantation of a cardiac resynchronization therapy device, such as the device illustrated in FIG. 1. Such surgical preparations are known in the art. Preparation of the patient may include a pre-operative visualization of the patient's heart using known techniques such as fluoroscopy, echocardiograms, CT scans, or the like. Once preparation is finalized, the surgeon places one or more cardiac resynchronization therapy pacing leads into the proper locations into the heart.

For proper cardiac resynchronization therapy pacing, the surgeon must implant at least one lead into the right ventricle (RV). The lead is placed within the right ventricle in a first position, and is capable of delivering cardiac resynchronization therapy pacing events, i.e. electrical stimulation, to the right ventricle. A second lead, which is electrically coupled to the left ventricle (LV), is placed in a first position, preferably in the cardiac sinus branch. Placement of the right ventricle and left ventricle leads, which provide pacing and sensing, can be performed with the aid of visualization. For example, fluoroscopy or echocardiograms can be used to aid the surgeon when placing the cardiac resynchronization therapy leads into their respective first positions. While placing the leads into such positions may be routine in the art, the instant invention provides techniques which provide the surgeon with data obtained from sensors that indicate proper placement of the leads. Proper placement of the leads allows the cardiac resynchronization therapy pacing event resynchronization of multiple chambers of the heart, such as the left ventricle and the right ventricle to provide electro and mechanical events at the correct time.

One of the difficult aspects of cardiac resynchronization therapy lead placement is determining where to place the leads. Methods currently in use are problematic and typically only provide a 2 dimensional image, often resulting in the leads being placed too closely together and in poor synchronization. Typical fluoroscopy provides the surgeon with visualization of the right ventricle and the left ventricle vasculature. However, because fluoroscopy only provides a 2D image, fluoroscopy fails to provide the surgeon with the ability to determine proper lead spacing. While the vasculature branches may appear to be close to the right ventricle when viewed under fluoroscopy, such spacing is not always correct, resulting in the surgeon placing the leads at various random points which are believed to be at a proper distance sufficient to provide synchronization. Because fluoroscopy does not give a true three-dimensional view, the actual distance may be closer or farther than what the surgeon sees on the screen. The instant invention overcomes such difficulties by providing cardiac resynchronization therapy pacing leads which include a sensor which can obtain data that provides efficiency determining characteristics. As described previously, sensors such as those manufactured by Mediguide can be used to obtain data, such as the physical distance between the right ventricle and the leads which pace and sense the left ventricle, such as the coronary sinus branch. The sensor should also be able to provide data with regards to the relative motion of both the right ventricle and left ventricle cardiac resynchronization therapy pacing leads.

Once the right and left ventricle pacing leads are placed within the proper first positions, cardiac resynchronization therapy efficiency characteristics are obtained for at least one cardiac cycle or rhythm. The cardiac resynchronization therapy efficiency characteristics, preferably the position of the leads and the relative motion of the leads, are observed, determined, or calculated for at least one cardiac cycle pacing event. Such data can be observed visually or preferably can be reported and processed by processing devices, such as computers and software programs. In a typical situation in which the heart is not synchronized, the two leads may move in a parallel manner or away from each other. After obtaining such information, the cardiac resynchronization therapy device provides electrical stimulation (cardiac resynchronization therapy pacing event). The cardiac resynchronization therapy efficiency characteristics, preferably the position of the leads and the relative motion of the leads, are observed, determined, or calculated for at least one cardiac cycle pacing event. Such data can be observed visually or preferably can be reported and processed by processing devices, such as computers, and software programs. If the placement of the right ventricle and the left ventricle pacing leads are proper, resulting in synchronization of the heart, no other steps are required. The patient undergoes post-operative procedures.

If however, the initial positioning of the leads is not correct, the surgeon must reposition one or both leads. As an illustrative example, the left ventricle cardiac resynchronization therapy pacing lead is moved from its first position to a second position. This second position is typically another area associated with the left ventricle vasculature, either some other position within the same vein or a different vein. Once in place, the cardiac resynchronization therapy device provides a cardiac resynchronization therapy pacing event and data related to cardiac resynchronization therapy efficiency characteristics are obtained. Specifically, the surgeon obtains the distance between the electrodes and the relative motions.

The surgeon is looking for data which indicates that the right ventricle and left ventricle are synchronized. This data includes the physical distance between the leads and the relative motion of the leads during right ventricular only pacing, left ventricular only pacing and cardiac resynchronization therapy biventricular pacing. If good lead positioning for both the right ventricular and left ventricular lead has been obtained, then the operator will be able to demonstrate that there is greater than a pre-defined minimal physical distance between the leads after implantation. In addition, good resynchronization will be associated with a change in the relative dynamic movement of the left ventricular free wall versus the right ventricular pacing lead and good resynchronization should be associated with movement of both leads towards each other, versus movement of the right ventricular and left ventricular lead away from each other should poor lead positioning have been obtained. This method will therefore allow the surgeon to demonstrate, in real time, that adequate lead separation has objectively and quantitatively been achieved and that resynchronization is occurring with these respective lead positions. If such data is obtained, the cardiac resynchronization therapy device has properly synchronized the heart and the surgeon is satisfied with the lead placement.

As illustrated in FIG. 3, a third lead, which although not required, may be placed near or within the right atria. If such lead is used, data from the procedure and data obtained for the left and right ventricle leads can be applied to the right atrium lead.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method for determining the efficacy of cardiac resynchronization pacing utilizing simultaneous right ventricle and left ventricle electroanatomic cardiac phase motion mapping comprising the steps of:
1) using at least 2 cardiac resynchronization therapy pacing leads each having one or more sensors for obtaining data related to lead placement efficiency determining characteristics into a patient's heart, said leads being electrically coupled to a cardiac resynchronization therapy pacing device for delivery of a cardiac resynchronization therapy event;
2) observing one or more efficiency determining characteristics for each of said leads for at least one cycle of said patient's cardiac cycle;
3) providing a pacing event to said heart from said cardiac resynchronization therapy pacing device;
4) observing one or more efficiency determining characteristics for each said lead for at least one cardiac cycle;
5) determining if said position of each said leads relative to each other provides efficient cardiac resynchronization therapy to achieve synchronized cardiac cycle;
6) moving at least one of said leads to a second position in the heart if said cardiac resynchronization therapy does not result in synchronized cardiac cycles;
7) providing a second pacing event to said at least one repositioned leads;
8) observing one or more efficiency determining characteristics for said repositioned leads for at least one said cardiac cycle;
9) determining if said repositioned leads provides efficient cardiac resynchronization therapy to achieve synchronized cardiac cycle;
10) repeating steps 6-9 until synchronized cardiac cycles are achieved.

2. The method according to claim 1 wherein at least one lead is electrically coupled to the right ventricle and at least one lead is electrically coupled to the left ventricle.

3. The method according to claim 1 further including using a third cardiac resynchronization therapy pacing lead, said lead having one or more sensors for obtaining data related to lead placement efficiency determining characteristics, said lead being electrically coupled to a cardiac resynchronization therapy pacing device for delivery of a cardiac resynchronization therapy event.

4. The method of according to claim 3 wherein said third lead is electrically coupled to the right atrium of said patient's heart.

5. The method according to claim 2 wherein said at least one lead is electrically coupled to said left ventricle and is placed in said coronary sinus branch.

6. The method according to claim 1 wherein said efficiency determining characteristics include the distance between said pacing leads.

7. The method according to claim 1 wherein said efficiency determining characteristics include the relative motion of at least one lead compared to the relative motion of at least a second lead.

8. The method according to claim 1 wherein said synchronized cardiac cycles are determined by observing said efficiency determining characteristics, wherein said at least two pacing leads move toward each other when a pacing event is started.

9. A method for determining the efficacy of cardiac resynchronization pacing utilizing simultaneous right ventricle and left ventricle electroanatomic cardiac phase motion mapping comprising the steps of:
introducing a first CRT lead having one or more sensors for obtaining cardiac resynchronization therapy efficiency characteristics, said first lead being electrically coupled to the right ventricle of a patient's heart;
introducing a second cardiac resynchronization therapy lead having one or more sensors for obtaining cardiac resynchronization therapy efficiency characteristics, said second lead being electrically coupled to the left ventricle of said patient's heart;
obtaining efficiency characteristics of the said first lead and said second lead prior to providing a CRT pacing event;
providing a cardiac resynchronization therapy pacing event;
obtaining a second set of efficacy determining characteristics of said first and second leads after said pacing event;
determining if the cardiac resynchronization therapy pacing event correctly resynchronized the patient's heart.

10. The method of claim 11 further including the steps of;
moving at least one lead to another position in the patient's heart if said cardiac resynchronization therapy pacing event did not result in resynchronized heart;
obtaining additional cardiac resynchronization therapy pacing efficacy characteristics for said reposition leads;
delivering an additional cardiac resynchronization therapy pacing event;
determining if repositioning of said lead(s) resulted in resynchronization of said patient's heart.

11. The method of claim 10 further including the steps of repeating one or more steps until each said lead is placed in a position which results in resynchronization of said patient's heart.

12. The method of claim 9 further including the step of positioning a third lead into the right atrium, said third lead having a sensor for obtaining data related to efficiency determining characteristics.

13. The method according to claim 9 wherein placement of said second lead includes the coronary sinus branch.

14. The method according to claim 11 wherein repositioning of said second lead includes placement within one or more vasculature components of the left ventricle.

15. The method according to claim 9 wherein said efficiency determining characteristics includes the distance between each said lead.

16. The method according to claim 9 wherein said efficiency determining characteristics include the relative motion of each said lead.
